(19) United States
(12) Patent Application Publication
(10) Pub. No.: US 2023/0197280 A1
(43) Pub. Date: Jun. 22, 2023

(54) B-AMYLOID POSITIVE CONVERSION TARGET PREDICTION DEVICE

(71) Applicant: SAMSUNG LIFE PUBLIC WELFARE FOUNDATION, Seoul (KR)

(72) Inventors: Sang Won SEO, Seoul (KR); Chae Jung PARK, Seoul (KR); Jun Pyo KIM, Yongin-si (KR)

(21) Appl. No.: 18/078,600

(22) Filed: Dec. 9, 2022

(30) Foreign Application Priority Data
Dec. 17, 2021 (KR) 10-2021-0181415

(51) Int. Cl. G16H 50/30; G16B 20/00

(52) U.S. Cl. CPC G16H 50/30; G16B 20/00

(57) ABSTRACT

A beta (p)-amyloid positive conversion target prediction device is provided. The β-amyloid positive conversion target prediction device includes: a patient information analyzer configured to provide first input information by differentiating age and gender of a β-amyloid negative patient based on basic information of a patient, a genotype analyzer configured to provide second input information for determining an apolipoprotein genotype status of the patient, a standardized update value ratio (SUVR) analyzer configured to provide an SUVR calculated from amyloid positron emission tomography (PET)test results of the patient as third input information and an artificial intelligence (AI) model configured to provide prediction results related to a β-amyloid positive conversion status of the β-amyloid negative patient based on at least one of the first input information, the second input information, and the third input information.

### CROSS-REFERENCE TO RELATED APPLICATION

This application claims the benefit of Korean Patent Application No. 10-2021-0181415 filed on Dec. 17, 2021, in the Korean Intellectual Property Office, the entire disclosure of which is incorporated herein by reference for all purposes.

### BACKGROUND

### 1. Field of the Invention

At least one example embodiment relates to a beta (β)-amyloid positive conversion target prediction device.

### 2. Description of the Related Art

Until recently, the treatment of Alzheimer’s disease mainly includes drug therapy, improvement of lifestyle, and cognitive training that aim at relieving symptoms for patients with already developed symptoms. Such treatment all has characteristics of delaying or relieving symptoms after the onset of the disease (β-amyloid cerebral deposition), that is, secondary or tertiary prevention. However, in the future, interest in a primary prevention aspect that may prevent the disease even before the deposition of beta (β)-amyloid protein is expected to increase and various studies related thereto are currently being conducted.

### SUMMARY

A technical objective of at least one example embodiment provides a beta (β)-amyloid positive conversion target prediction device capable of early predicting a potential patient that may develop Alzheimer’s disease by providing prediction results related to a β-amyloid positive conversion status of a p-amyloid negative patient using an artificial intelligence (AI) model that is pretrained based on at least one of first input information corresponding to age and gender of the β-amyloid negative patient, second input information regarding whether the β-amyloid negative patient has an apolipoprotein genotype (apolipoprotein epsilon 4 (APOE4) genetic information), and third input information corresponding to a standardized uptake value ratio (SUVR).

In an embodiment, a beta (β)-amyloid positive conversion target prediction device may include a patient information analyzer configured to provide first input information by differentiating age and gender of a β-amyloid negative patient based on basic information of a patient, a genotype analyzer configured to provide second input information for determining an apolipoprotein genotype status of the patient, a standardized update value ratio (SUVR) analyzer configured to provide an SUVR calculated from amyloid positron emission tomography (PET)test results of the patient as third input information and an artificial intelligence (AI) model configured to provide prediction results related to a β-amyloid positive conversion status of the β-amyloid negative patient based on at least one of the first input information, the second input information, and the third input information.

In some embodiments, the patient information analyzer may include a patient classifier configured to classify the β-amyloid negative patient and a β-amyloid positive patient based on the basic information of the patient.

In some embodiments, the SUVR analyzer may include a global value ratio provider configured to provide a global SUVR calculated from a global region of an amyloid PET video and a regional value ratio provider configured to provide a regional SUVR calculated from each of partial regions of the amyloid PET video.

In some embodiments, the third input information may include the global SUVR and the regional SUVR.

In some embodiments, the SUVR analyzer may include a region selector configured to select a portion of the partial regions and to provide the regional SUVR corresponding to a selection region.

In some embodiments, the β-amyloid positive conversion target prediction device may further include a weight provider configured to provide an input weight to be applied to each of the first input information, the second input information, and the third input information.

In some embodiments, the weight provider may include a selection weight provider configured to provide a selection weight applied to the regional SUVR corresponding to the selection region.

In some embodiments, the β-amyloid positive conversion target prediction device may further include a result determiner configured to determine that the β-amyloid negative patient has a β-amyloid positive conversion probability when a value of the prediction results is greater than a preset positive conversion reference value.

In some embodiments, the β-amyloid positive conversion target prediction device may further include a display configured to display the prediction results and the β-amyloid positive conversion probability of the β-amyloid negative patient.

In another embodiment, an operation method of a beta (β)-amyloid positive conversion target prediction device may include: providing, by a patient information analyzer, first input information by differentiating age and gender of a β-amyloid negative patient based on basic information of a patient, providing, by a genotype analyzer, second input information for determining an apolipoprotein genotype status of the patient, providing, by a standardized update value ratio (SUVR) analyzer, an SUVR calculated from amyloid positron emission tomography (PET) test results of the patient as third input information and providing, by an artificial intelligence (AI) model, prediction results related to a β-amyloid positive conversion status of the β-amyloid negative patient based on at least one of the first input information, the second input information, and the third input information.A β-amyloid positive conversion target prediction device according to example embodiments may early predict a potential patient that may develop Alzheimer’s disease by providing prediction results related to a β-amyloid positive conversion status of a β-amyloid negative patient using an AI model that is pretrained based on at least one of first input information corresponding to age and gender of the β-amyloid negative patient, second input information regarding whether the β-amyloid negative patient has an apolipoprotein genotype, and third input information corresponding to an SUVR.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 is a diagram illustrating a beta (β)-amyloid positive conversion target prediction device according to example embodiments;

FIG. 2 is a diagram illustrating an example of a patient information analyzer included in the β-amyloid positive conversion target prediction device of FIG. 1;

FIG. 3 is a table for describing an operation of a patient classifier included in the patient information analyzer of FIG. 2;

FIG. 4 is a diagram illustrating an example of a standardized uptake value ratio (SUVR) analyzer included in the β-amyloid positive conversion target prediction device of FIG. 1;

FIG. 5 illustrates an example for describing an operation of the SUVR analyzer of FIG. 4;

FIG. 6 is a diagram illustrating another example of the SUVR analyzer included in the β-amyloid positive conversion target prediction device of FIG. 1;

FIG. 7 is a diagram for describing a weight provider included in the β-amyloid positive conversion target prediction device of FIG. 1;

FIGS. 8 and 9 illustrate examples for describing operations of the weight provider of FIG. 7;

FIG. 10 is a diagram for describing a result determiner and a display included in the β-amyloid positive conversion target prediction device of FIG. 1; and

FIG. 11 is a flowchart illustrating an operation method of a β-amyloid positive conversion target prediction device according to example embodiments.

### DETAILED DESCRIPTION

In example embodiments, advantages, features, and methods for achieving example embodiments will become more apparent after a reading of the following exemplary embodiments taken in conjunction with the drawings. The example embodiments may, however, be embodied in different forms and should not be construed as being limited to the embodiments set forth herein. Rather, these embodiments are provided to describe the present invention in detail to the extent that a person skilled in the art to which the invention pertains can easily enforce the technical concept of the present invention.

It is to be understood herein that embodiments of the present invention are not limited to the particulars shown in the drawings and that the drawings are not necessarily to scale and in some instances proportions may have been exaggerated in order to more clearly depict certain features of the invention. While particular terminology is used herein, it is to be appreciated that the terminology used herein is for the purpose of describing particular embodiments only and is not intended to limit the scope of the present invention.

As used herein, the term “and/or” includes any and all combinations of one or more of the associated listed items. It will be understood that when an element is referred to as being “on,” “connected to” or “coupled to” another element, it may be directly on, connected or coupled to the other element or intervening elements may be present. As used herein, a singular form is intended to include plural forms as well, unless the context clearly indicates otherwise. It will be further understood that the terms “includes ” and/or “including,” when used in this specification, specify the presence of at least one stated feature, step, operation, and/or element, but do not preclude the presence or addition of one or more other features, steps, operations, and/or elements thereof.

Hereinafter, example embodiments will be described in detail with reference to the accompanying drawings.

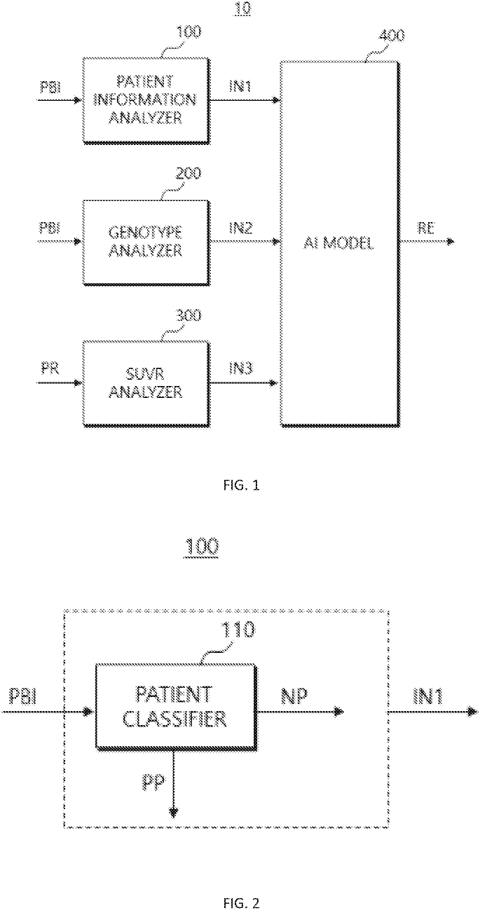

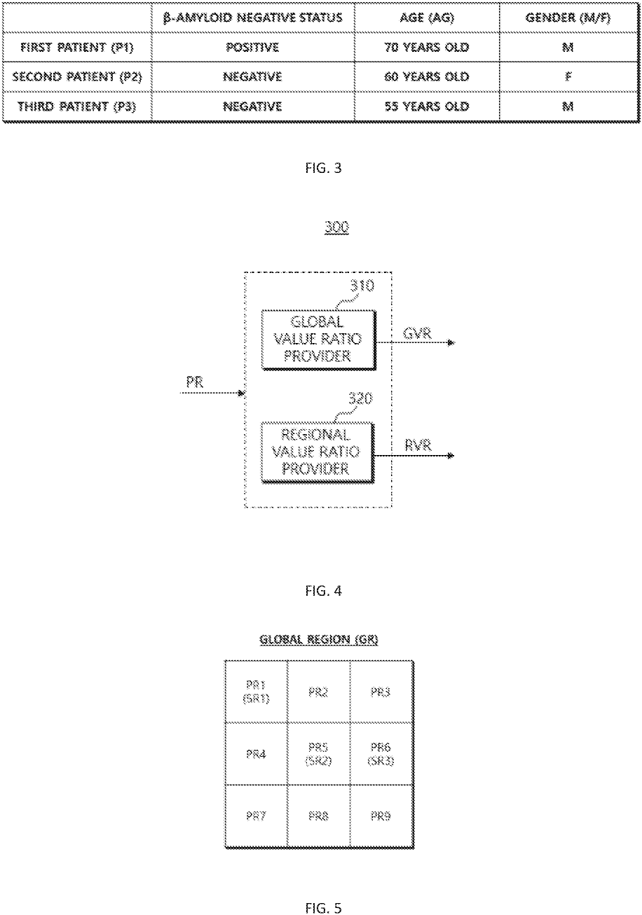

FIG. 1 is a diagram illustrating a beta (β)-amyloid positive conversion target prediction device according to example embodiments, FIG. 2 is a diagram illustrating an example of a patient information analyzer included in the β-amyloid positive conversion target prediction device of FIG. 1, and FIG. 3 is a table for describing an operation of a patient classifier included in the patient information analyzer of FIG. 2.

Referring to FIGS. 1 to 3, a β-amyloid positive conversion target prediction device 10 according to an example embodiment may include a patient information analyzer 100, a genotype analyzer 200, a standardized update value ratio (SUVR) analyzer 300, and an artificial intelligence (AI) model 400. The patient information analyzer 100 may provide first input information (IN1) by differentiating age (AG) and gender (M/F) of a β-amyloid negative patient (NP) based on basic information (PBI) of a patient. For example, the basic information (PBI) of the patient may include information on a β-amyloid negative status, an apolipoprotein genotype status (apolipoprotein epsilon 4 (APOE4) genetic information), age (AG), and gender (M/F) of the patient.

In an example embodiment, the patient information analyzer 100 may include a patient classifier 110. The patient classifier 110 may classify a β-amyloid negative patient (NP) and a β-amyloid positive patient (PP) based on basic information of a corresponding patient (PBI). For example, a plurality of patients may include a first patient (P1), a second patient (P2), and a third patient (P3). The first patient (P1) may be a 70-year-old male and may be β-amyloid positive, and the second patient (P2) may be a 60-year-old female and may be β-amyloid negative. Also, the third patient (P3) may be a 55-year-old male and may be β-amyloid negative. In this case, the patient classifier 110 may provide age (AG) and gender (M/F) in basic information (PBI) of the second patient (P2) and basic information (PBI) of the third patient (P3) that are β-amyloid negative, as the first input information (INI).

The genotype analyzer 200 may provide second input information (IN2) for determining an apolipoprotein genotype status of the patient, that is, whether the patient has an apolipoprotein genotype. For example, the genotype analyzer 200 may determine the apolipoprotein genotype status (apolipoprotein epsilon 4 (APOE4) genetic information) based on basic information (PBI) of the patient and may provide the second input information (IN2) for determining the apolipoprotein genotype status. The apolipoprotein genotype may be associated with the pathogenesis of Alzheimer’s disease.

The SUVR analyzer 300 may provide an SUVR calculated from amyloid positron emission tomography (PET) test results (PR) of the patient as third input information (IN3). The AI model 400 may provide prediction results related to a β-amyloid positive conversion of the β-amyloid negative patient (NP) based on at least one of the first input information (IN1), the second input information (IN2), and the third input information (IN3). The AI model 400 may be in a state in which a sufficient amount of the first input information (IN1) to the third input information (IN3) on various patients are learned before the β-amyloid positive conversion target prediction device 10 according to an example embodiment operates.

The β-amyloid positive conversion target prediction device 10 according to an example embodiment may early predict a potential patient that may develop Alzheimer’s disease by providing the prediction results (RE) related to a β-amyloid positive conversion status of the β-amyloid negative patient (NP) using the AI model 400 that is pretrained based on at least one of the first input information (IN1) corresponding to age (AG) and gender (M/F) of the β-amyloid negative patient (NP), the second input information (IN2) regarding whether the β-amyloid negative patient (NP) has an apolipoprotein genotype, and the third input information (IN3) corresponding to an SUVR.

FIG. 4 is a diagram illustrating an example of an SUVR analyzer included in the β-amyloid positive conversion target prediction device of FIG. 1, FIG. 5 illustrates an example for describing an operation of the SUVR analyzer of FIG. 4, and FIG. 6 is a diagram illustrating another example of the SUVR analyzer included in the β-amyloid positive conversion target prediction device of FIG. 1.

Referring to FIGS. 1 to 6, the SUVR analyzer 300 may include a global value ratio provider 310 and a regional value ratio provider 320. The global value ratio provider 310 may provide a global SUVR (GVR) calculated from a global region (GR) of an amyloid PET video (test results). The regional value ratio provider 320 may provide a regional SUVR (RVR) calculated from each of partial regions (PR1 to PR9) of the amyloid PET video.

For example, the global region (GR) may refer to a region that represents the entire brain captured as the PET video and regions obtained by dividing the global region (GR) into a plurality of regions may be the partial regions (PR1 to PR9). An SUVR corresponding to the global region (GR) may be a global SUVR (GVR) and an SUVR corresponding to the partial region may be a regional SUVR (RVR). The global SUVR (GVR) and the regional SUVR (RVR) may be calculated from amyloid PET test results (PR).

Also, for example, the plurality of partial regions (PR1 to PR9) divided from the global region (GR) may include a first partial region (PR1) to a nineth partial region (PR9). Regional SUVRs (RVRs) respectively corresponding to the first partial region (PR1) to the nineth partial region (PR9) may include a first RVR regional SUVR (RR1) to a nineth regional SUVR (RR9). In an example embodiment, the third input information (IN3) may include the global SUVR (GVR) and the regional SUVR (RVR).

In an example embodiment, the SUVR analyzer 300 may further include a region selector 330. The region selector 330 may select a portion of the partial regions (PR1 to PR9) and may provide a regional SUVR (RVR) corresponding to a selection region (SR). The region selector 330 may be used to calculate prediction results (RE) using only some regional SUVRs (RVRs) among the plurality of regional SUVRs (RVRs). For example, the plurality of partial regions (PR1 to PR9) may include the first partial region (PR1) to the nineth partial region (PR9). The region selector 330 may select the first partial region (PR1) as a first selection region (SRI) and may select the fifth partial region (PR5) as a second selection region (SR2) according to a selection signal (SS). Also, the region selector 330 may select the sixth partial region (PR6) as a third selection region (SR3). In this case, the regional value ratio provider 320 may provide the first regional SUVR (RR1) corresponding to the regional SUVR (RVR) of the first partial region (PR1), the fifth regional SUVR (RR5) corresponding to the regional SUVR (RVR) of the fifth partial region (PR5), and the sixth regional SUVR (RR6) corresponding to the regional SUVR (RVR) of the sixth partial region (PR6) to the AI model 400.

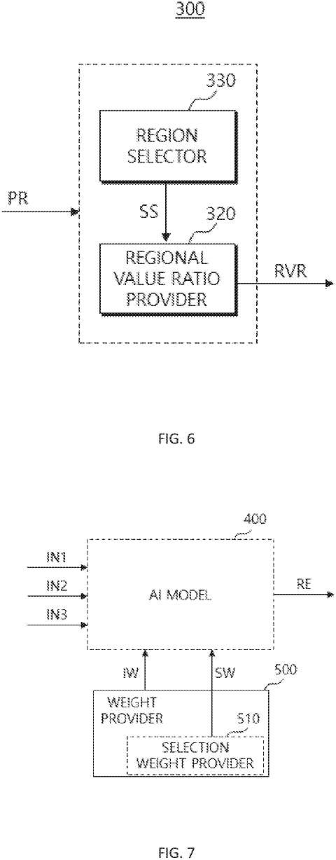

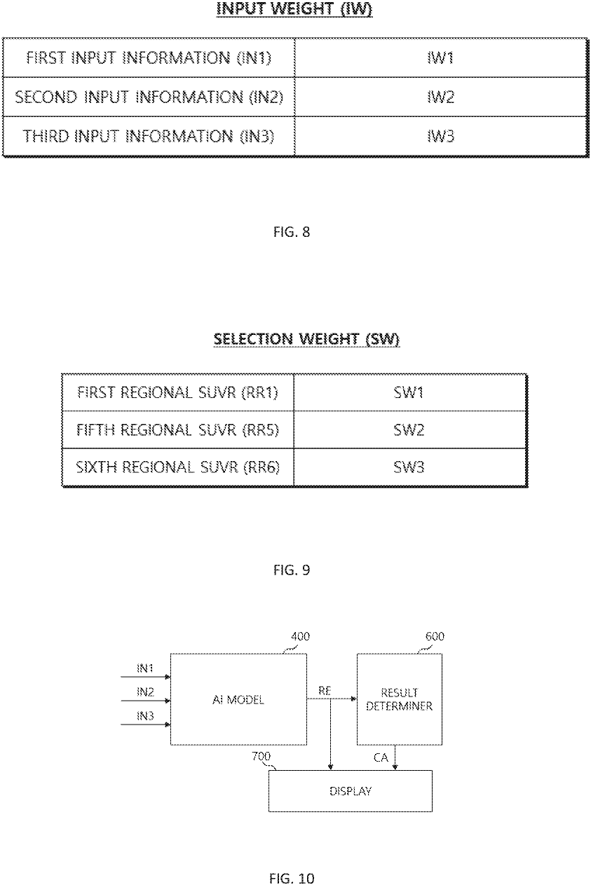

FIG. 7 is a diagram for describing a weight provider included in the β-amyloid positive conversion target prediction device of FIG. 1, FIGS. 8 and 9 illustrate examples for describing operations of the weight provider of FIG. 7, and FIG. 10 is a diagram for describing a result determiner and a display included in the β-amyloid positive conversion target prediction device of FIG. 1.

Referring to FIGS. 1 to 10, in an example embodiment, the β-amyloid positive conversion target prediction device 10 may further include a weight provider 500. The weight provider 500 may provide an input weight (IW) to be applied to each of the first input information (IN1), the second input information (IN2), and the third input information (IN3).

For example, a user using the β-amyloid positive conversion target prediction device 10 according to an example embodiment may determine that importance is high in order of the third input information (IN3), the second input information (IN2), and the first input information (IN1) among the first input information (IN1), the second input information (IN2), and the third input information (IN3). In this case, the weight provider 500 may provide a first input weight (IW1) as the input weight (IW) corresponding to the first input information (IN1) and may provide a second input weight (IW2) as the input weight (IW) corresponding to the second input information (IN2). Also, the weight provider 500 may provide a third input weight (IW3) as the input weight (IW) corresponding to the third input information (IN3). Here, the third input weight (IW3) may be greater than the second input weight (IW2) and the second input weight (IW2) may be greater than the first input weight (IW1).

In an example embodiment, the weight provider 500 may further include a selection weight provider 510. The selection weight provider 510 may provide a selection weight (SW) to be applied to a regional SUVR (RVR) corresponding to a selection region. For example, the region selector 330 may select the first partial region (PR1) as the first selection region (SR1) and may select the fifth partial region (PR5) as the second selection region (SR2) according to a selection signal (SS). Also, the region selector 330 may select the sixth partial region (PR6) as the third selection region (SR3). In this case, the regional value ratio provider 320 may provide the first regional SUVR (RR1), the fifth regional SUVR (RR5), and the sixth regional SUVR (RR6) to the AI model 400. The user using the β-amyloid positive conversion target prediction device 10 according to an example embodiment may determine that importance is high in order of the first regional SUVR (RR1), the fifth regional SUVR (RR5), and a sixth regional SUVR (RR6) among regional SUVRs (RVRs) included in the third input information (IN3). In this case, the selection weight provider 510 may provide a first selection weight (SW1) as the selection weight (SW) corresponding to the first regional SUVR (RR1) and may provide a second selection weight (SW2) as the selection weight (SW) corresponding to the fifth regional SUVR (RR5). Also, the weight provider 500 may provide a third selection weight (SW3) as the selection weight (SW) corresponding to the sixth regional SUVR (RR6). Here, the first selection weight (SW1) may be greater than the second selection weight (SW2) and the second selection weight (SW2) may be greater than the third selection weight (SW3).

In an example embodiment, the β-amyloid positive conversion target prediction device 10 may further include a result determiner 600. When a value of the prediction results (RE) is greater than a preset positive conversion reference value, the result determiner 600 may determine that the β-amyloid negative patient (NP) has a β-amyloid positive conversion probability (CA). For example, the result determiner 600 may provide the β-amyloid positive conversion probability (CA) of the β-amyloid negative patient (NP) as a probability of developing Alzheimer’s disease within 5 years. Also, the p-amyloid positive conversion target prediction device 10 may further include a display 700. For example, the display 700 may display the prediction results (RE) and the β-amyloid positive conversion probability (CA) of the β-amyloid negative patient (NP).

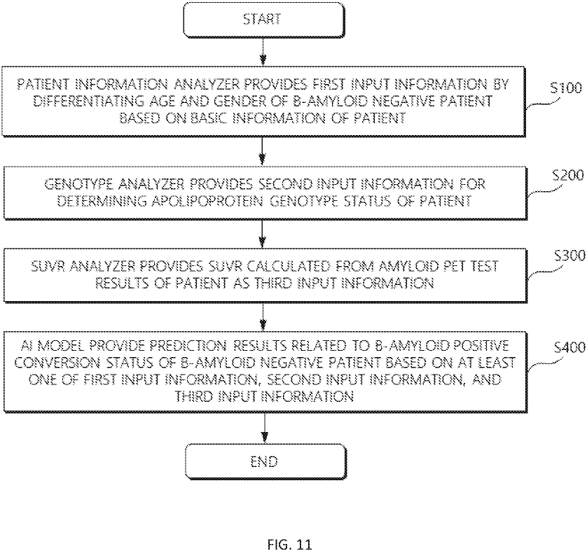

FIG. 11 is a flowchart illustrating an operation method of a β-amyloid positive conversion target prediction device according to example embodiments.

Referring to FIGS. 1 to 11, in the operation method of the β-amyloid positive conversion target prediction device 10 according to an example embodiment, the patient information analyzer 100 may provide first input information (IN1) by differentiating age (AG) and gender (M/F) of a β-amyloid negative patient (NP) based on basic information (PBI) of a patient in operation S100. In operation S200, the genotype analyzer 200 may provide second input information (IN2) for determining an apolipoprotein genotype status of the patient. In operation S300, the SUVR analyzer 300 may provide an SUVR calculated from amyloid PET test results (PR) of the patient as third input information (IN3). In operation S400, the AI model 400 may provide prediction results (RE) related to a β-amyloid positive conversion status of the β-amyloid negative patient (NP) based on at least one of the first input information (IN1), the second input information (IN2), and the third input information (IN3).

The β-amyloid positive conversion target prediction device 10 according to an example embodiment may early predict a potential patient that may develop Alzheimer’s disease by providing the prediction results (RE) related to a β-amyloid positive conversion status of the β-amyloid negative patient (NP) using the AI model 400 that is pretrained based on at least one of the first input information (IN1) corresponding to age (AG) and gender (M/F) of the β-amyloid negative patient (NP), the second input information (IN2) regarding whether the β-amyloid negative patient (NP) has an apolipoprotein genotype, and the third input information (IN3) corresponding to an SUVR.

In concluding the detailed description, those skilled in the art will appreciate that many variations and modifications may be made to the preferred embodiments without substantially departing from the principles of the present invention. Therefore, the disclosed preferred embodiments of the invention are used in a generic and descriptive sense only and not for purposes of limitation.

What is claimed is:

1. A beta (β)-amyloid positive conversion target prediction device comprising:
a patient information analyzer configured to provide first input information by differentiating age and gender of a β-amyloid negative patient based on basic information of a patient;
a genotype analyzer configured to provide second input information for determining an apolipoprotein genotype status of the patient;
a standardized update value ratio (SUVR) analyzer configured to provide an SUVR calculated from amyloid positron emission tomography (PET)test results of the patient as third input information; and
an artificial intelligence (AI) model configured to provide prediction results related to a β-amyloid positive conversion status of the β-amyloid negative patient based on at least one of the first input information, the second input information, and the third input information.

2. The β-amyloid positive conversion target prediction device of claim 1, wherein the patient information analyzer comprises a patient classifier configured to classify the β-amyloid negative patient and a β-amyloid positive patient based on the basic information of the patient.

3. The β-amyloid positive conversion target prediction device of claim 2, wherein the SUVR analyzer comprises:
a global value ratio provider configured to provide a global SUVR calculated from a global region of an amyloid PET video; and
a regional value ratio provider configured to provide a regional SUVR calculated from each of partial regions of the amyloid PET video.

4. The β-amyloid positive conversion target prediction device of claim 3, wherein the third input information includes the global SUVR and the regional SUVR.

5. The β-amyloid positive conversion target prediction device of claim 4, wherein the SUVR analyzer further comprises a region selector configured to select a portion of the partial regions and to provide the regional SUVR corresponding to a selection region.

6. The β-amyloid positive conversion target prediction device of claim 5, further comprising:
a weight provider configured to provide an input weight to be applied to each of the first input information, the second input information, and the third input information.

7. The β-amyloid positive conversion target prediction device of claim 6, wherein the weight provider comprises a selection weight provider configured to provide a selection weight applied to the regional SUVR corresponding to the selection region.

8. The β-amyloid positive conversion target prediction device of claim 7, further comprising:
a result determiner configured to determine that the β-amyloid negative patient has a β-amyloid positive conversion probability when a value of the prediction results is greater than a preset positive conversion reference value.

9. The β-amyloid positive conversion target prediction device of claim 8, further comprising:
a display configured to display the prediction results and the β-amyloid positive conversion probability of the β-amyloid negative patient.

10. An operation method of a beta (β)-amyloid positive conversion target prediction device, the method comprising:
providing, by a patient information analyzer, first input information by differentiating age and gender of a β-amyloid negative patient based on basic information of a patient;
providing, by a genotype analyzer, second input information for determining an apolipoprotein genotype status of the patient;
providing, by a standardized update value ratio (SUVR) analyzer, an SUVR calculated from amyloid positron emission tomography (PET) test results of the patient as third input information; and
providing, by an artificial intelligence (AI) model, prediction results related to a β-amyloid positive conversion status of the β-amyloid negative patient based on at least one of the first input information, the second input information, and the third input information.
